(19) United States
(12) Patent Application Publication
(10) Pub. No.: US 2020/0170568 A1
(43) Pub. Date: Jun. 4, 2020

(54) Wearable Electronic System

(71) Applicant: DREEM, Paris (FR)

(72) Inventors: Hugo MERCIER, Paris (FR); Quentin SQULET DE BRUGIERE, Paris (FR); Clémence PINEAUD, Paris (FR); David DEHAENE, Paris (FR); Artémis LLAMOSI, Paris (FR); Gabriel OPPETIT, Paris (FR); Pierrick ARNAL, Saint Mande (FR)

(73) Assignee: DREEM, Paris (FR)

(21) Appl. No.: 16/621,017

(22) PCT Filed: Jun. 11, 2018
(86) PCT No.: PCT/EP2018/065370

§ 371 Date: Dec. 10, 2019

(30) Foreign Application Priority Data
Jun. 12, 2017 (FR) 17 55221

(51) Int. Cl. A61B 5/00; A61M 21/02; A61B 5/0484

(52) U.S. Cl. CPC A61B 5/4809; A61B 5/6802; A61B 5/4812; A61M 2230/06; A61B 5/04845; A61M 2230/205; A61M 2230/42; A61M 21/02

(57) ABSTRACT

A wearable electronics system for helping a user fall asleep comprising: a wearable portion; an electronic portion comprising: an acquisition module for making a physiological measurement of the user; a playing module for playing an acoustic stimulation to the user; a memory storing a plurality of elemental acoustic stimulation sequences; a processing module comprising a selection module which selects elemental acoustic stimulation sequences depending on a history of previously played acoustic stimulations and depending on the physiological measurement, until the end of the period of falling asleep The acquisition module and the playing module are assembled in the wearable portion.

The invention relates to the field of wearable electronics systems.

More precisely, the invention relates to the field of wearable electronics systems for helping a user fall asleep.

Sleep is considered an important time during which an individual can recover. Recently, several wearable electronic devices were brought up as aiming to improve the sleep of an individual. This works with the measurement of a physiological signal of the individual and by playing signals which could change the sleep of the individual, prepared from the measured physiological signals. “Physiological signal” is understood to mean any signal arising from the activity of the individual.

WO 2016/083,598 is interested in the simulation of slow brain waves and more specifically the deep sleep phase of the individual. This document therefore describes the simulation of an individual while sleeping.

WO 2017/021,662 describes the customization of a method for acoustic stimulation of brain waves of an individual. This method is mainly implemented during periods of sleep.

WO 2015/087,188 describes a method for transition between different phases of sleep. It is explicitly indicated that the stimulation is played at an appropriate time for avoiding interfering with the phase of falling asleep.

The WO 2012/106,444 and WO 2015/013,576 family has a different approach because they aim to improve the quality of sleep by thermal regulation.

Unlike the systems presented above, the invention aims to improve the falling asleep of the user.

The invention is disclosed below.

According to a first aspect, the object of the invention is a wearable electronics system for helping a user fall asleep comprising:
a wearable portion suited to clothe the user;
an electronic portion comprising, functionally connected to each other;
   at least one acquisition module suited for making a physiological measurement of the user;
   at least one playing module suited for playing an acoustic stimulation to the user;
   a memory storing a plurality of elemental acoustic stimulation sequences;
   a processing module comprising a selection module suited for, repetitively during a period of falling asleep, selecting at least one elemental acoustic stimulation sequence from the plurality of elemental acoustic stimulation sequences stored in memory, depending on a history of previously played acoustic stimulations and depending on the physiological measurement, where the playing module is ordered to play said acoustic stimulation sequence, until the end of the period of falling asleep;
      where at least the acquisition module and the playing module are assembled in the wearable portion.

With these provisions, the user is guided to falling asleep.

According to an embodiment, the end of the period of falling asleep is determined by an end determination module of the processing module based on the physiological measurement.

According to an implementation, the electronic portion generates, by repeated use of the selection module, an acoustic stimulation comprising a temporal succession of acoustic stimulation sequence signals, and in which the temporal succession is determined by the selection module depending on the history of previously played acoustic stimulations and/or depending on the physiological measurement.

According to an embodiment, a composition module generates an acoustic stimulation sequence signal based on a preset acoustic stimulation sequence template coming from a template database.

According to an embodiment, the composition module generates an acoustic stimulation sequence signal by superposition of several elemental acoustic stimulation sequences.

According to an embodiment, the elemental acoustic stimulation sequences are generated from databases of long length sound tracks, short length sounds, words or scenes, and phrases.

According to an embodiment, the wearable electronics system comprises a module for pairing sound tracks, sounds, words or scenes.

According to an embodiment, the wearable electronics system further comprises a portable computer proposing an interface interacting with the processing module.

According to an embodiment, the acquisition module is suited for making a physiological measurement of an acceleration, a movement, a heartbeat, a breathing cycle, a blood oxygen saturation or an electroencephalogram of the user.

According to an embodiment, the selection module selects an elemental acoustic stimulation sequence based on a falling-asleep metric estimator determined by a falling-asleep metric determination module based on the physiological measurement.

According to an embodiment, a target state indicator is determined, and the selection module selects an elemental acoustic stimulation sequence from a database relating a possibility of reaching the targeted state indicator for the user and the elemental acoustic stimulation sequences.

The figures from the drawings are now briefly described.

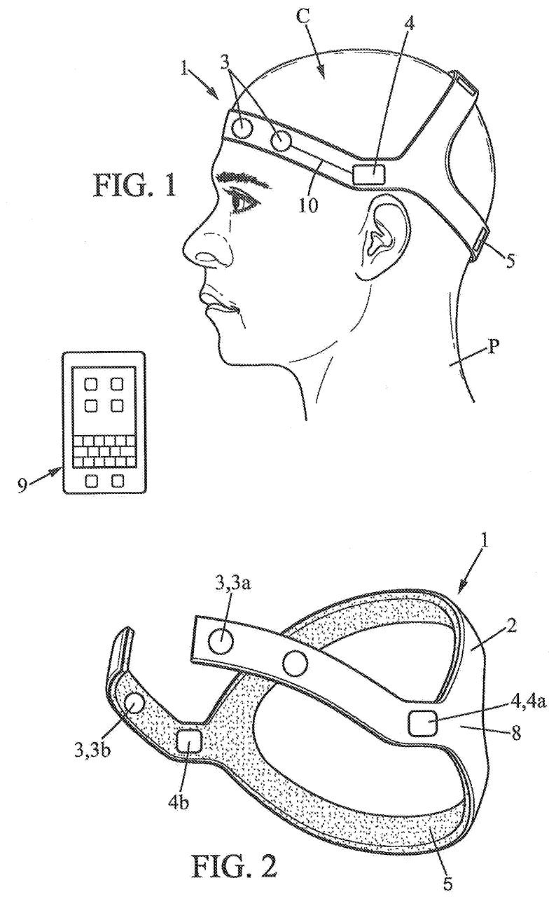

FIG. 1 is a schematic view of an acoustic stimulation device for an individual according to an embodiment of the invention;

FIG. 2 is a detailed view in perspective of the device from FIG. 1 in which the device in particular comprises a first and the second acoustic transducer respectively suited for emitting acoustic signals stimulating respectively a right inner ear and a left inner ear of the individual.

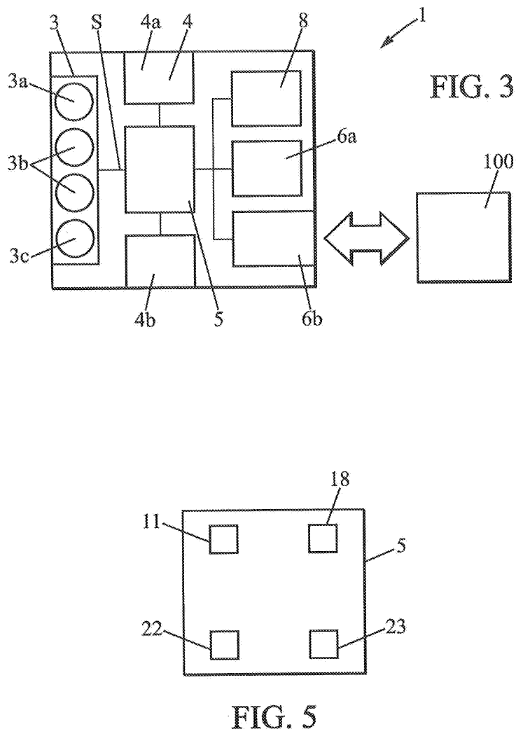

FIG. 3 is an overview drawing of the system according to a first embodiment of the invention comprising a device;

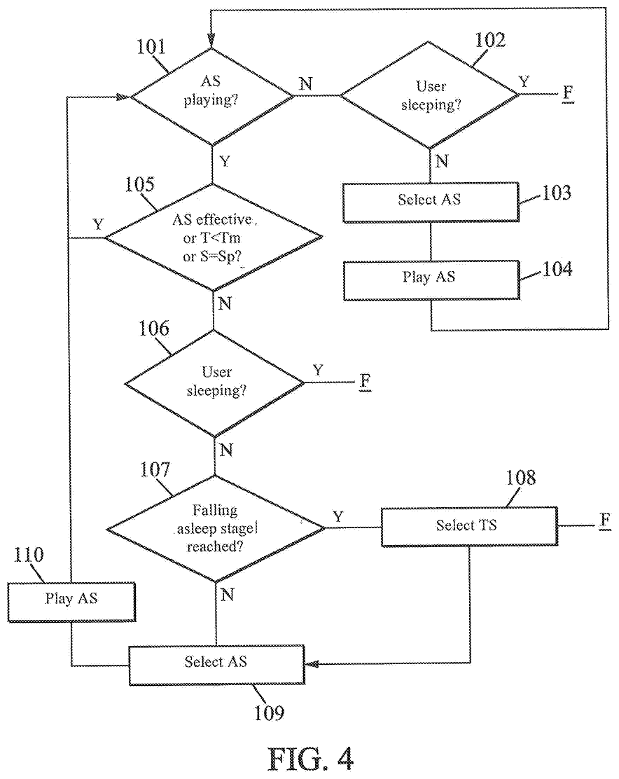

FIG. 4 in a diagram representative of an embodiment;

FIG. 5 is a detailed schematic view of the processing module according to an implementation example;

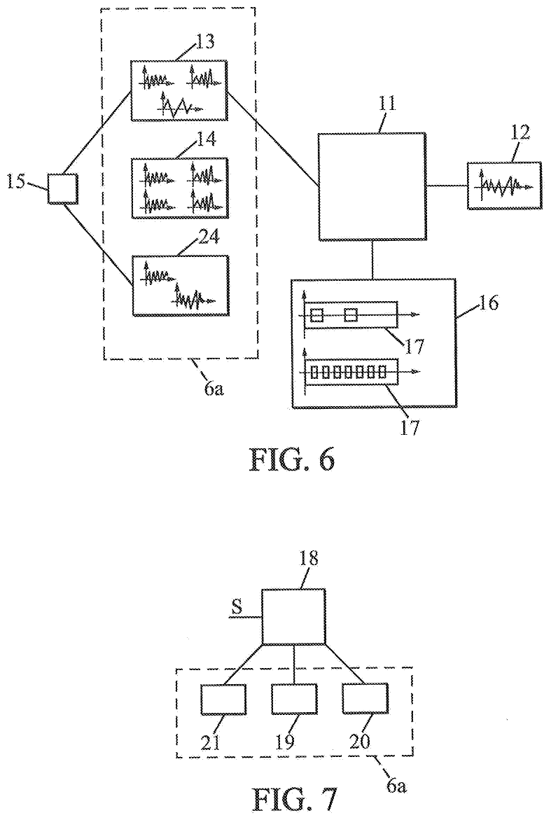

FIG. 6 is a detailed schematic view of the composition module according to an implementation example; and

FIG. 7 is a detailed schematic view of the falling-asleep metric determination module according to an implementation example.

In the various figures, the same references designate identical or similar items.

The object of the invention is a system 1 for acoustic stimulation of the individual P, which is shown in FIGS. 1 to 7 and allowing implementation of an acoustic stimulation method.

As shown in FIGS. 1 to 3, the system 1 comprises a module for acquisition 3 of at least one measured signal, a processing module 5 and a module for playing 4 a signal from an acoustic stimulation sequence signal.

A part at least of the system 1 can be wearable on the head of the individual P, for example at least the acquisition module 3.

In an embodiment of the invention, the system 1 is at least partially wearable by the individual P, for example on the head of the individual P.

For this purpose, the system 1 can comprise one or more support elements 2 able to surround at least partially the head of the individual P so as to be held there. The support elements 2 for example take the shape of one or more branches which can be arranged so as to surround the head of the individual P to keep the elements of the system 1 there. The support elements 2 thus form a wearable portion clothing the user.

The system 1 can also be divided into one or more elements suited to be worn on different parts of the body of the individual P, for example on the head, wrist or even on the torso. The various elements then communicate with each other by wire or wirelessly (in which case the various elements are equipped with wireless transmitters and/or receivers for data transfer).

The system 1 can comprise a user input interface allowing the user to configure the system 1. As applicable, the system 1 comprises an electronic interface component, such as a portable computer 9 communicating wirelessly or by wire with the processing module 5. The portable computer 9 can execute a computer program allowing the user to enter configuration data and send them to the processing module 5. With the interface, the user can select a functionality for help falling asleep from a predefined list (examples of functionality for falling asleep are described below). Some parameters can also be set via the interface, for example one or more themes (for the soundtracks, sounds and/or words), a maximum length, etc. A parameter can be to select only words from the sounds. “Random” can also be chosen in place of a given theme. These parameters can depend on the chosen functionality, or not.

As shown on FIG. 5, the processing module 5 comprises a composition module 11 suited for generating an acoustic stimulation from databases of more elementary signals. The processing module 5 further comprises a falling-asleep metric determination module 18 for determining the falling-asleep state of the user. The processing module 5 comprises a selection module 22 suited for generating a scenario to help falling asleep comprising acoustic stimulations that can be generated by the composition module 11 where the scenario takes the falling-asleep metric into account. The processing module 5 comprises an end determination module 23 suited for ending the acoustic stimulation.

The method according to the invention comprises a step of supplying an acoustic stimulation sequence signal.

An acoustic stimulation sequence signal can in particular be made as a composite signal resulting from the superposition of two or more elemental acoustic stimulation sequence signals.

Each elemental acoustic stimulation sequence signal is representative of a continuous complex acoustic signal.

“Continuous complex acoustic signal” is understood to mean an acoustic signal comprising at least two distinct acoustic frequencies and extending over a time greater than one or more seconds.

An example of continuous complex acoustic signal is music or chant, or even a natural sound, meaning a sound generated without human intervention, such as rain, wind, waves, bird songs, etc. Another example of complex acoustic signal is white or pink noise. Another example of continuous complex acoustic signal is, for example, a syllable, a word, a series of words, a phrase.

A continuous complex acoustic signal is therefore very different from a single sinusoid or short pulse.

A continuous complex acoustic signal has in particular specific auditory properties and in particular a significant listening comfort which allows listening to such a sound for long times of several hours without annoyance.

“The acoustic stimulation sequence signal is representative of a continuous complex acoustic signal” means that the acoustic stimulation sequence signal is representative of said acoustic stimulation signal suited for being played by a sound playing device. Each elemental acoustic stimulation sequence signal is thus for example a digital recording, compressed or uncompressed, of said acoustic signal, meaning a series of numbers coding for the acoustic signal in a way suitable for being stored or manipulated by electronic components like memory and processors, while also being suited to be translated into acoustic waves by a sound transducer.

Each elemental acoustic stimulation sequence signal is for example a recording in a file format in WAV, OOG or AIF format.

According to the invention, a database is provided which stores a large number of different elemental acoustic stimulation sequences.

The elemental acoustic stimulation sequences can be classified according to the type thereof.

In particular, according to the invention, at least three different types of elemental acoustic stimulation sequences can be considered.

A first type of elemental acoustic stimulation sequence is that of soundtracks. A soundtrack is a fairly long acoustic signal (typically longer than one minute), and relatively repetitive nature, such as music, a long natural sound such as noise from a wave, rain, rustling leaves, which could be cyclic, etc.

A second type of elemental acoustic stimulation sequence is that of sounds. A sound is a fairly short acoustic signal, of order one second, and of rather weakly repetitive nature, such as a word. Among the sounds, cognitive sounds are distinguished; without being articulated words, these refer to a cognitive concept for the user. Among the sounds, a subtype is called “words,” meaning articulated sounds having an intelligible meaning for the user. Among the sounds, another subtype is called “scenes,” or groups of sounds, grouped together according to a cognitive logic (meaning that the sounds put together have some meaning, involving either words, or sounds having a cognitive content). Thus, in the above description, only the word “word,” the word “scene,” or the expression “cognitive sound” is sometimes used in a context where any one of these words or expressions could be used, except that it follows from the context that the words or expressions “words,” “cognitive sounds” and “scenes” are used in contrast to each other.

A third type of elemental acoustic stimulation sequence is that of phrases. A phrase is a fairly intermediate length acoustic signal, of order several seconds, and of rather weakly repetitive nature. Further, a phrase has an intelligible meaning for the user. Acoustically, phrases are little different from scenes. But for use of the system, they are used in a preliminary phase of instructions to give the user instructions instead of during an effective phase.

The elemental acoustic stimulation sequences can also be classified according to the theme thereof. This is the case in particular of the soundtracks and/or the sounds. “Theme” is understood to mean a cognitive theme for the user. Examples of cognitive themes include, for example but without limitation, forest, wind, rain, sand, sea, wave, animals, etc. A single elemental acoustic stimulation sequence can be classified with several themes.

A composition module 11 serves to generate an acoustic stimulation sequence 12 from elemental acoustic stimulation sequences from the database. The base of an acoustic stimulation sequence comprises a soundtrack, which has a certain length, and a certain amplitude (as applicable, variable over time), coming from the soundtrack database 13. The composition module 11 assigns a start time for the soundtrack. The composition module 11 composes the soundtrack with a sound, which has some length, and some amplitude (as applicable, variable over time), coming from the sound database 14. The composition module 11 assigns a start time for the sound relative to the start time for the soundtrack. The composition module 11 also determines an amplitude ratio between the soundtrack and the sound during the length of the sound. As applicable, the composition module 11 assigns several sounds during the length of the soundtrack. In this case, the composition module 11 assigns the starting times for each sound and the relative amplitude thereof. Further, the composition module 11 can select the sounds and soundtracks on the basis of the theme thereof. For example, the composition module 11 can select all the sounds corresponding to a single theme. The composition module 11 can select the soundtrack and the sounds corresponding to a single theme. Additionally or alternatively, the composition module 11 composes the soundtrack with a phrase, which has some length, and some amplitude (as applicable, variable over time), coming from the phrase database 24. The composition module 11 assigns a start time for the phrase relative to the start time for the soundtrack. The composition module 11 also determines an amplitude ratio between the soundtrack and the phrase during the length of the phrase. As applicable, the composition module 11 assigns several phrases during the length of the soundtrack, and/or both at least one phrase and one sound. In this case, the composition module 11 assigns the starting times for each phrase and the relative amplitude thereof. Generally, a phrase and a sound are not superposed at the same moment.

The composition module can generate an acoustic stimulation sequence 12 based on a preset acoustic stimulation sequence template 17 coming from a template database 16. A preset acoustic stimulation sequence template 17 can comprise one or more phrases at certain preset moments of the acoustic stimulation sequence, and also rules for selecting soundtracks, sounds, and the moments, themes and amplitudes thereof.

Thus, when speaking of the superposition (two or more) of several elemental acoustic simulation sequences (soundtracks, phrases and/or sounds), it involves a mechanism by which these elemental acoustic stimulation sequences are played simultaneously, which goes through a management of the amplitude of each of the elemental acoustic stimulation sequences in order to not increase a general amplitude of the acoustic stimulation signal played relative to the preceding or following moments where fewer elemental acoustic stimulation sequences are used.

A pairing module 15 can be provided for combining two (or more) elemental acoustic stimulation sequences in order for one or more functionalities or for one or more phases of functionalities. For example, several soundtracks, several sounds, several words, one soundtrack and one word/sound, etc. can be paired with each other. The pairs are generated randomly or depending on cognitive and/or acoustic criteria. For example, the pairs are generated depending on themes of elemental acoustic simulation sequences. The paired elemental acoustic stimulation sequences are assigned a single label.

The system 1 can comprise a storage module 6a for elemental acoustic stimulation sequences, for example a memory 6a suited for storing a portion or all of the elemental acoustic stimulation sequence signals.

The storage module 6a can for example be a removable module, for example a memory card such as an SD card (acronym for “Secure Digital”), or else a memory permanently mounted in the system 1.

The storage module 6a can further be suited for storing the various variables in quantities mentioned in the remainder of the description and used by the method according to the invention, for example a measurement signal S, elemental acoustic stimulation sequence signals, acoustic stimulation sequence signals, etc.

The system 1 can also comprise a communication module 6b for communicating with a server 100 for retrieving elemental acoustic stimulation sequence signals from said server 100 by complete downloading or by streaming.

The server 100 can be a computer, but also a smart phone, a smart watch, or another microcontroller or microprocessor. As applicable, it is the same as the portable computer 9.

For this purpose, the communication module 6b can be able to communicate with said server 100 directly, via a local or wide area network like the Internet.

The communication module 6b can communicate with said server 100 via a wired connection, for example by USB, or a wireless connection, for example a Wi-Fi and/or Bluetooth connection. Other communication protocols can obviously be used.

The method according to the invention can further comprise a step of falling-asleep metric determination for the user. This step goes through the acquisition of at least one measurement signal S by means of the acquisition module 3.

The measurement signal S can in particular be representative of a physiological electrical signal E of the individual P.

The physiological electrical signal E can for example comprise an electroencephalogram (EEG), an electromyogram (EMG), an electrooculogram (EOG), an electrocardiogram (ECG) or any other measurable biosignal of the individual P. As a variant or in addition, it may comprise a blood oxygen saturation signal, as obtained from a pulse oximeter. As a variant or in addition, it may involve a signal representative of the movement, as obtained from one or more inertial sensors. A measurement of the temperature, or mechanical waves (sounds, vibrations, etc.) of the user are also conceivable. Preferably, in the context of the invention, the measurements that are the least invasive possible for the user are preferred.

For this purpose, according to an example, the acquisition module 3 comprises a plurality of electrodes 3 suitable for being in contact with the individual P, and notably with the skin of the individual P for acquiring at least one measurement signal S representative of a physiological electrical signal E of the individual P.

The physiological electrical signal E advantageously comprises an electroencephalogram (EEG) of the individual P.

For this purpose, in an embodiment of the invention, the system 1 comprises at least one EEG measurement electrode 3b. In a preferred embodiment, the system 1 comprises at least two electrodes 3 including at least one reference electrode 3a and at least one EEG measurement electrode 3b.

The system 1 can further comprise one bulk ground electrode 3c.

In a specific embodiment, the system 1 comprises at least three EEG measurement electrodes 3c, so as to acquire physiological electrical signals E comprising at least three electroencephalogram measurement channels.

The EEG measurement electrodes 3 are for example arranged on the surface of the skin of the skull of the individual P, notably on the surface of the scalp and/or the forehead of the individual P.

In other embodiments, the system 1 can further comprise an EMG measurement electrode and, possibly, an EOG measurement electrode.

The measurement electrodes 3 can be reusable electrodes or disposable electrodes. Advantageously, the measurement electrodes 3 are reusable electrodes so as to simplify the daily use of the system.

The measurement electrodes 3 can be, notably, dry electrodes or electrodes covered with a contact gel. The electrodes 3 can also be textile, silicone or polymer electrodes.

The acquisition module 3 can also comprise measurement signal S acquisition devices that are not solely electric.

A measurement signal S can thus be, generally, representative of a physiological signal of the individual P.

The measurement signal S can also comprise a sub-signal representative of a physiological signal of the individual P that is not electric or not completely electric, for example a signal of cardiac activity, such as a heart rhythm, a breathing rate and/or depth, a body temperature of the individual P or even movements of the individual P, mechanical waves (e.g. sounds, vibrations, etc.) of the individual P, or even measurements of the response of the individual P to a stimulus, as obtained for example by functional near-infrared spectroscopy (“FNIR”).

For this purpose, the acquisition module 3 can comprise a heart rhythm detector, a body thermometer or even a three-axis accelerometer.

The acquisition module 3 can again comprise measurement signal S acquisition devices representative of the environment of the individual P.

The measurement signal S can thus further comprise a sub-signal representative of the quality of the air around the individual P, for example carbon dioxide or oxygen level, or even a temperature or a noise signal, notably a sub-signal representative of an ambient noise level.

In an embodiment of the invention, the step of acquisition of the measurement signal S further comprises a pre-processing of the measurement signal S.

Thus, according to the invention, a falling-asleep metric determination module 18 for the user determines a falling-asleep metric for the user from at least the measurement signal S acquired by the acquisition module 3.

According to a first exemplary implementation, the falling-asleep metric determination module 18 determines a falling-asleep estimator from the measurement signal S representative of the heart rhythm of the user measured by the acquisition module 3. The acquisition module 3 then comprises at least one heart rate meter. For example, the measurement signal F representative of the cardiac rhythm of the user is compared with a bank of reference data representative of heart rhythms while falling asleep, and the falling-asleep estimator is determined from this comparison. According to a specific example, a bank of reference data representative of the heart rhythm comprises one or more heart rhythm thresholds each associated with a value for the falling-asleep estimator. The comparison of the measurement signal S representative of the heart rhythm of the user with this or these thresholds serves to characterize the degree of falling asleep of the user.

The description which was just given can also alternatively apply to other measurement signals. For example, the description which was just given can apply to the variability of the heart rate. Use of any relevant parameter obtained from measuring the heart rhythm can be considered.

Alternatively, the description which was just given can also apply to the breathing rhythm. The acquisition module 3 then comprises at least one accelerometer, in particular a three-axis accelerometer. The signals from the accelerometer can be processed by the method of decomposition into principal components. Along the three axes, a sinusoidal type component having an amplitude in a predetermined range with frequency of order the breathing frequency is then observed. As a variant or in addition, the pulse oximeter is used for determining the breathing rhythm.

Alternatively, the description which was just given can also apply to the variability of the breathing rhythm. Use of any relevant parameter obtained from measuring the breathing rhythm can be considered.

Alternatively, the description which was just given can also apply to the movements of the user. The acquisition module 3 then comprises at least one accelerometer. Notably, if the acquisition module 3 is worn on the head of the user, it is not influenced much by breathing movements, such that the detected movements correspond to macroscopic movements of the user. The frequency of the detected acceleration signal, and in particular a low-frequency representative of the time separation between two macroscopic movements of the user can be compared with a bank of reference data representative of the low frequency while falling asleep. As a variant, another relevant representative part coming from the acceleration signal can be used, such as, for example, the amplitude or any other relevant parameter resulting from the acceleration signal.

Alternatively, the description which was just given can also apply to the electroencephalogram of the user. The acquisition module 3 then comprises at least the EEG measurement electrodes described above. The falling-asleep metric determination module can extract representative brain wave patterns from the measured signal. This step can for example be done, using a bank of reference data 19 representative of brain wave patterns, by comparing the measured signal in this bank and by extracting the parts of the measured signal representing those stored in the bank. Then, the extracted parts of the signal are compared to a database 20 of representative signals associated with a falling-asleep estimator. For example, the database in question comprises EEG signals associated with sleep stages classified according to the Hod classification.

An alternative method for determination of the falling-asleep estimator from EEG signals considers one and/or another of the beta, alpha or theta waves of the user.

A first step comprises the analysis of the spectral content of the measurement signal S for defining an actual spectral content ASC indicator.

In order to implement this analysis, the measurement signal S can in particular be stored in a memory, for example in a buffer memory.

To do that, a spectrum of the measurement signal is determined, for example by implementing a Fourier transformation or wavelet transformation.

Next, the spectrum of the measurement signal is analyzed in order to define an actual spectral content ASC indicator.

“Actual spectral content indicator” is understood to mean one or more values calculated from the spectrum of the measurement signal, or from the measurement signal, and serving to quantify the spectral content of the measurement signal. An actual spectral content indicator can for example comprise a central frequency of the measured signal, a frequency range comprising a majority of the energy of the measured signal, or even an energy weighting of one or more frequencies or one or more frequency ranges of the measured signal.

Such an actual spectral content indicator ASC can for example indicate that the measurement signal S is representative of brain waves in a frequency range included between 10 Hz and 40 Hz with a central frequency of about 16 Hz.

The actual spectral content ASC indicator is for example calculated regularly over time, according to a sliding time window. The representative signal of the actual spectral content ASC indicator over time is for example compared to a bank of reference data 21 representative of spectral content indicator signals over time representative of falling asleep. For example, we want to identify a pattern over time comprising a decrease in beta waves (12-40 Hz), followed by an increase of alpha waves (8-12 Hz), then an increase of theta waves (4-8 Hz). Such a pattern can be seen to give a high score for the falling asleep indicator. Other processing is conceivable based on the spectral content of the EEG signal. For example, a ratio between the spectral power density of each of the aforementioned waves can be measured. Alternatively, an amplitude ratio of the determined maximum peak for each of the two frequency bands can be measured.

In the preceding examples, the falling-asleep indicator can be determined based on one or another of these approaches. Alternatively, in the preceding examples, the falling-asleep indicator can be determined based on a function of one or another of these approaches.

Again another alternative is to determine a falling-asleep indicator based on two of the approaches described above. As appropriate, considering more than two of these approaches is conceivable.

The falling-asleep indicator can be customized for the user. Meaning that the rule assigning a falling-asleep indicator depending on the measured signals can be personal to the user. For example, if the user subsequently provides information about their previously falling asleep (for example, the next morning the user provides information about falling asleep the past night) to the falling-asleep metric determination module 18 via the interface, this information can be considered by the falling asleep metric determination module 18 for adapting the falling-asleep indicator determination rules.

The falling-asleep indicator can serve to classify the falling-asleep state of the user into various classes. For example, the following different classes are conceivable:
the user has a chance of falling asleep;
the user is not falling asleep;
the user is in a preset phase of falling asleep, among a plurality of preset phases of falling asleep;
the user is at the beginning of the NREM1 stage of falling asleep,
the user is at the beginning of the NREM2 stage of falling asleep.

If the user has fallen sufficiently asleep (for example because they are in the beginning of stage NREM2), as determined from the falling-asleep indicator, the end determination module 23 can terminate the acoustic stimulation.

During the second sub-step, it is determined whether, depending on the falling-asleep metric, an acoustic stimulation must be continued, started or interrupted. The falling-asleep metric indicator is compared with the target state.

The target state is representative of a targeted falling-asleep state of the user. The target state indicator is not calculated from the measurement signal S but is preset, and for example stored in the memory 6a of the system 1.

An exemplary implementation is described below in connection with FIG. 4. According to a first example, in step 101 it is determined whether an acoustic stimulation is already in progress.

If an acoustic stimulation is not already in progress (arrow N coming from box 101), it is determined whether it is necessary to start an acoustic stimulation. For that, the falling-asleep metric indicator is compared with the target state in step 102. The result of this comparison (arrow Y coming from box 102) can define whether it is necessary to start an acoustic stimulation. This is the case for example if it is determined that the user is in a sufficiently advanced state of falling asleep.

Alternatively, the result of this comparison (arrow Y coming from box 102) can also define that it is not necessary to start an acoustic stimulation, if it is determined that the user is not sufficiently far along in falling asleep.

If it is determined (arrow N coming from box 102) that the user may benefit from an acoustic stimulation, the selection module 22 then selects an acoustic stimulation sequence (step 103) to play to the user. The acoustic simulation sequence can notably be selected depending upon one or another of:
User data prestored by the user;
Prestored adjustment of the system (meaning that the system can store, as determined in advance, a sequence of two or more acoustic stimulation sequences or acoustic stimulation templates);
Falling-asleep metric indicator;
Targeted state indicated;
History of acoustic stimulation sequences played over a given prior period, for example since the beginning of the night, or considering the history of one or more previous nights.

As explained above, the selection of an acoustic stimulation sequence by the selection module 22 involves the selection of one or more elemental acoustic stimulation sequence(s) composing the acoustic stimulation sequence.

The playing module 4 then plays the acoustic stimulation sequence (step 104).

Now returning to step 101 for determining whether an acoustic stimulation step is already in progress, previously mentioned, it is possible that the result of this determination is positive (arrow Y coming from the box 101). In this case, it is determined whether it is necessary to continue, modify or stop the acoustic stimulation in progress. For that, the falling-asleep metric indicator is compared with the target state indicator (step 105).

One result of this comparison can be that it is necessary to continue the acoustic stimulation (arrow Y coming from box 105). For that, the comparison can also consider the history of the falling-asleep metric indicator. If the comparison determines that the falling-asleep metric indicator is progressing towards the targeted state indicator since a prior moment of the acoustic stimulation, but remains far from the targeted state indicator, the comparator can decide to continue the acoustic stimulation (arrow Y coming from box 105).

One result of this comparison can be that it is necessary to change the acoustic stimulation. For that, the comparison can also consider the history of the falling-asleep metric indicator. If the comparison determines that the falling-asleep metric indicator is not making sufficient progress towards the targeted state indicator since a prior moment of the acoustic stimulation, the comparator can decide to change the acoustic stimulation. The method can then go towards a step for selection of an acoustic stimulation sequence (arrow N coming from box 105).

According to another example, if the comparison determines that the falling-asleep metric indicator has sufficiently progressed towards the target state indicator since a previous time in the acoustic stimulation, such that the acoustic stimulation in progress is no longer effective, the method can then go towards a step of selection of an acoustic stimulation sequence (arrow N coming from box 105). First, perhaps the user is sleeping (step 106), and in that case (arrow Y coming from box 106), the end determination module 23 terminates the acoustic stimulation. Otherwise (arrow N coming from box 106), the comparator can decide to change the acoustic stimulation and the target state indicator. In practice, changing the acoustic stimulation is considered appropriate for guiding the user to a falling-asleep state either because the preceding stimulation was fruitful, or because it was ineffective for too long. To start, it is determined whether it is necessary to select a new target state indicator (step 107). The method then determines (arrow Y coming from box 107), a new target state indicator (step 108). The new target state indicator can in particular be selected depending upon one and/or another of:
User data prestored by the user;
Prestored adjustment of the system;
Falling-asleep metric indicator;
Indicator of the previous targeted content;
History of acoustic stimulation sequences over a given prior period, for example since the beginning of the night, or considering the history of one or more previous nights.

The method can then return (arrow coming from step 108) to step 109 for selection of an acoustic stimulation sequence, such as previously described. Or, if it is determined that there is no new target state indicator which could serve to guide the user to sleep, the end determination module 23 terminates the acoustic stimulation.

In step 107, it can also be determined to change the acoustic stimulation without changing the target state indicator. In this case (arrow N coming from box 107), it goes directly to step 109. The playing module 4 then plays the acoustic stimulation sequence (step 110).

It then returns to step 101.

As a variant, it goes to the step 109 of changing acoustic stimulation not after a determination based on the falling-asleep metric indicator, but, for example, at the end of a preset time determined by clock (step 105). As a variant, it goes to the step 109 of changing acoustic stimulation following recognition of a specific preset form in the physiological signal (step 105). To do that, a database of predetermined specific forms of the physiological signal Sₚ is available and a comparison module compares the measured signal with this database in order to recognize a specific form.

In this specific case, the subsequent acoustic stimulation sequence can be connected with the immediately preceding acoustic stimulation sequence. In particular, when the immediately preceding acoustic stimulation sequence comprises a compound signal resulting from the composition of two or more elemental acoustic stimulation sequences, the subsequent acoustic stimulation sequence can be an acoustic stimulation sequence comprising one or more elemental acoustic stimulation sequences from the immediately preceding acoustic stimulation sequence but in smaller number. As a variant, the subsequent acoustic stimulation sequence can comprise the same elemental acoustic stimulation sequences as the immediately preceding acoustic stimulation sequence, but in a different ratio. As it relates to the different ratios, it can for example be a matter of the respective amplitude of the elemental acoustic stimulation sequences which changes. Alternatively or in addition, it can also be a matter of a frequency of one of the elemental acoustic stimulation sequences which changes. “Frequency” is understood to mean the repetition frequency of the elemental acoustic stimulation sequence and not the frequency of the acoustic signal itself, which would affect the audibility of the elemental acoustic stimulation sequence.

If it is determined to stop the acoustic stimulation, it can also be done spread over time, for example by progressively reducing the amplitude of the acoustic stimulation sequence.

For this purpose, the playing module 4 is designed to play acoustic signals audible by the individual.

The playing module 4 thus comprises at least two acoustic transducers 4a, 4b.

A first acoustic transducer 4a is configured for stimulating mostly one from a right inner ear RE and a left inner ear LE of the individual P. A second acoustic transducer 4b is configured for stimulating mostly the other from a right inner ear RE and a left inner ear LE of the individual P.

In a first embodiment shown notably in FIGS. 1, 2 and 3, the acoustic transducers 4a, 4b are osteophonic devices stimulating the inner ears LE, RE of the individual P by bone conduction.

These osteophonic devices 4a, 4b can for example be suited for being placed near the ears, for example as shown in FIG. 1, in particular in an area of skin covering a cranial bone.

In a second embodiment, the acoustic transducers 4a, 4b are loudspeakers stimulating the inner ears LE, RE of the individual P by auditory conduits leading to said inner ears.

These loudspeakers can be arranged outside the ears of the individual P or in the auditory conduits.

In an embodiment of the invention shown in FIG. 4, the loudspeakers are separated from the remainder of the system 1 and for example arranged in the part in which the individual P is located.

In another embodiment of the invention, the acquisition module 3, the playing module 4 and the processing module 5 are mounted on the support elements 2 so as to be close to each other, such that the communication between these elements 3, 4 and 5 is particularly quick and high data rate.

To allow the implementation of the method according to the invention, the acquisition module 3, the playing module 4 and the processing module 5 are further functionally connected to each other and able to exchange information and commands.

Thus, for example, a maximum distance between the acquisition module 3, the processing module 5 and/or, as applicable, the playing module 4 can be less than a few meters and for example less than a few tens of centimeters. In this way, a sufficiently quick communication between the elements of the system 1 can be guaranteed.

The acquisition module 3, the processing module 5 and/or, as applicable, the playing module 4 can for example be housed in cavities of the support element 2, clipped onto the support element 2, or even again fixed to the support element 2 for example by adhering, screwing or any other suited attachment means. In an embodiment of the invention, the acquisition module 3, the processing module 5 and/or, as applicable, the playing module 4 can be mounted removably on the support element 2.

In an embodiment of the invention, the processing module 5 is functionally connected to the acquisition module 3 and to the playing module 4 via wired connections 10. In this way, the exposure of the individual P to electromagnetic radiation is reduced.

Further, the system 1 can also comprise a battery 8. The battery 8 can be mounted on the support element 2 as described above for the acquisition module 3 and the processing module 5. The battery 8 can in particular be able to supply the acquisition module 3 and the processing module 5. The battery 8 is preferably able to supply energy over several hours without recharging, preferably at least eight hours so as to cover an average sleeping time of an individual P.

In this way, the system 1 can operate autonomously for an extended operating time.

In this way in particular, the system 1 is autonomous and able to implement an operation for stimulation of brain waves without communicating with an external server, notably without communicating with an external server over several minutes, preferably several hours, preferably at least eight hours.

“Autonomous” is thus understood to mean that the system can, for example, operate for an extended period, several minutes, several hours, for example at least eight hours, without needing to be recharged with electric energy, to communicate with external elements such as the remote server or even to be structurally connected to an external device like an attachment element such as the arm of a stand.

In this way, the system can be used in the daily life of an individual P without imposing specific constraints.

Below, several implementation examples are given.

### Example 1: Cognitive Jamming

Starting:

The user starts their night with the “cognitive jamming” functionality as falling-asleep functionality. This functionality was prestored via the portable computer 9.

The user chooses the theme through the user interface. Beyond the various themes offered, the user can also choose “random”; this is the one by which the selection module 22 randomly selects the theme from the themes stored in the database. The theme is assigned both to the tracks and to the sounds. In particular, the words or scenes are selected from the sounds.

A soundtrack connected with the theme starts after a few seconds. This soundtrack will last through the entire functionality (one can conceive of a soundtrack of shorter length than the functionality, which will be played several times in a row). A phrase of voice instructions is superposed on the soundtrack. For example, the following phrase of instructions is superposed: “Let your imagination go free in the environment provided for you. Think of the scene, as it appears to you, without making a specific effort.”

Phase 1: Theme Words

A random succession of words connected with the selected theme (for example “nature”) is played, superposed on the soundtrack. The random words are played with an average spacing of a few seconds. The spacing might not be constant. It can be partially random. This phase lasts a preset maximum time, for example between 5 minutes and 30 minutes (if the functionality is not interrupted).

Phase 2: Transition to Multi-Theme Random Words:

Following this first phase of “Thematic words,” after a predetermined time a transitional phase starts, during which, for a predetermined time (for example from 2 to 10 minutes, and notably from 2 to 4 times less than the preceding phase), there are more and more multi-theme random words which are played. The words remain superposed with the soundtrack. On the one hand, the proportion of multi-theme random words among the words played is greater than that from phase 1. Further, this proportion increases over time during phase 2. The multi-theme random words are notably chosen randomly in the word database, after excluding the words from the theme, without consideration of the theme of the word. Thus, during this phase, it is determined that n words are going to be played during this time, where each of the n words are either from the theme or outside the theme, and the proportion of words from the theme decreases with time.

The time between words can also increase during this transition phase.

Phase 3: Multi-Theme Random Words

At the end of this transition phase, after a preset time, only multi-theme random words are played. The words are played superposed on the soundtrack. During a preset time (for example of intermediate length between the length of the transition phase and the length of the first phase), multi-theme random words are played. The time between words can increase during this phase.

As a variant, the sounds played are scenes in the place of words. Next words could be played with the progression of the functionality.

As a variant, the words can be combined pairwise (for example the average time between two successive words can altemate between a short average time (for example an average of order 3 seconds) and a long average time (for example an average of order 10 seconds), in order to push the user to make associations between the words, in order to be more immersive. In this case, the system comprises a pairing module 15 suited for bringing the words together by pairs. This pairing module 15 can be executed at the start-up of the functionality, and select the pairs of words randomly so as to be different from one execution to another. In this case, the pairing module adds a label to each word of a single pair and the labels are stored so as to allow subsequent pairing of the words. In particular, a first pairing is done among the words from the theme. A second pairing is done among the words outside the theme. During execution of the functionality, when a first word of a pair is selected randomly, the subsequent word played is the other word of the same pair. Alternatively, during execution of the functionality, instead of selecting words randomly, pairs of words are selected randomly and the words of a single pair are always played in a preset order.

As a variant, the words combined pairwise can be pairs built in advance.

In the above example, the movement between two successive acoustic stimulation sequences is done depending on a predetermined time. As a variant, this movement could further, or alternatively, take into consideration the falling-asleep metric indicator and the target state indicator.

As a variant, during the later use of the system (for example during a later night), the transition to later phases, compared to the previous night, is anticipated (whether by the time adjustment or depending on the falling-asleep metric). In fact, with use, the user is conditioned such that the transition to later phases guides them to falling asleep. Thus a neuronal feedback to the user (or, according to the term commonly used in the art, “neurofeedback”) is provided.

Output Ports.

At the end of functionality, or if the falling-asleep metric determination module determines that the user has a chance of falling asleep, the time between two successive words can be greatly increased in order to release the mental attention that is called for from the user and allow the user an opportunity to fall asleep. At the end of a predetermined time in this phase, if the falling-asleep metric determination module determines that the user is not falling asleep, the time between successive words can be shortened.

Evoked Potential.

According to a variant, the physiological signal is analyzed for determining the influence of the acoustic stimulation on falling asleep. This can notably be the case when the physiological signal is an electroencephalogram. It is recognized that an evoked potential can be generated and measured in the electroencephalogram shortly (for example about 50 to 100 ms) after acoustic stimulation and that such an evoked potential is short, for example of order 30 to 70 ms. By analyzing the evoked potential, it can be determined whether an acoustic stimulation signal has a beneficial or harmful effect on falling asleep. Based on this determination, subsequent use of this acoustic stimulation signal can be changed. Thus, the acoustic stimulation signal playing rule uses the analysis of the measured physiological signal in response to the prior playing of identical or similar acoustic stimulation signals (in particular with the same theme).

As a variant, cognitive sounds related to the theme could be used in place of words. As a variant, words, sounds or scenes can be played randomly.

As a variant, resuming at the same area of functionality could be considered in case of short pause, shorter than a preset time.

As a variant, the introductory phase is not played again if the functionality is restarted.

### Example 2: Relaxing Sounds

This functionality is similar to example 1 above, replacing words with sounds relating to cognitive content.

The method described above can be applied by using sounds relating to cognitive content in place of words. The various variants described for the first example also apply to this example.

### Example 3: Neuronal Feedback (or, According to the Term Commonly Used in the Art, “Neurofeedback”)

Starting the Functionality:

The user starts their night with the “neurofeedback” functionality as falling-asleep functionality.

The user chooses a sound theme that will be played (nature, space, sea, etc.); the sound theme defines both the first soundtrack played at the beginning of the night and the second soundtrack which will be played later.

A soundtrack for the theme starts after a few seconds. Voice instructions are superposed on the soundtrack. For example, the following phrase is played: “Relax and let yourself be carried away by listening to the theme that you chose. It will change with your brain activity.”

Transition Between Two Soundtracks

The first soundtrack is played so long as the falling-asleep metric determination module 18 does not measure the beginning of falling asleep of the individual. Depending on the progress of the falling-asleep state of the individual, a second soundtrack is superposed on the first soundtrack. In a later phase, depending on the progress of the falling-asleep state of the individual, the first soundtrack is stopped and the second soundtrack replaces it completely.

Several variants are conceivable for playing this transition.

According to a first aspect, the two soundtracks are not chosen randomly, but are selected to allow this superposition. One option, for that, is to prepare the first soundtrack as the superposition of one elemental soundtrack and a second elemental soundtrack and to prepare the second soundtrack as the superposition of the same first elemental soundtrack and a third elemental soundtrack.

According to another variant of the above aspect, the second and third elemental soundtracks are replaced by a second set of successive sounds (between spaces of silence) and a third set of successive sounds (between spaces of silence), respectively.

According to another variant, the transition between the first and second soundtrack is done via intermediate soundtracks played between the first soundtrack and the second soundtrack. The passage from an earlier soundtrack to a later soundtrack can be done based on the determination of a falling asleep metric or according to a preset sequence. For example, the soundtracks closest to the first or second soundtrack are repeated more often than the soundtracks further from both the first and second soundtrack. The passage from one soundtrack to a later soundtrack can include a temporal superposition of the two soundtracks.

The variants described in the above examples are applicable here.

### Example 4: Free Breathing

Starting the Functionality:

The user starts their night with the “free breathing” functionality as falling asleep functionality, and chooses a theme (e.g. nature).

A base soundtrack connected with the theme starts after a few seconds.

A phrase of vocal instructions is sent, superposed on the base soundtrack. For example, the following phrase is played: “Focus on your breathing, and breathe normally.”

Synchronization of the Sound with the Breathing

For some time, the base soundtrack is played. During this time, signals representative of the user's breathing are measured. At the end of some time, based on measured signals, representative parameters of the user's breathing are determined. The soundtrack is played for example for the time to get a measurement of one or more parameters representative of the breathing. In particular a control module can be used which compares estimated parameters representative of the breathing with the database, so as to confirm the measurements, or to call them into question based on the correspondence between the estimated representative parameters and the database.

Based on the measured physiological signals, parameters representative of the user's breathing, for example an approximate rate and approximate phase over time, are determined. Once the breathing is thus detected, a back-and-forth soundtrack is generated based on parameters representative of the breathing. A back-and-forth soundtrack is a pseudo-periodic soundtrack pseudo-periodic around a given frequency. Such a back-and-forth soundtrack is typically a wave noise. A back-and-forth soundtrack with frequency similar to the determined breathing frequency is normally chosen. The back-and-forth soundtrack is played in synchronization with the inhalation and exhalation and superposed on the base soundtrack. The approximate phase of the breathing is also used such that the back-and-forth soundtrack is substantially in phase with the breathing. The first back-and-forth sound of the back-and-forth soundtrack which is played can start at the trough of the breathing (end of exhalation moment) in order to avoid an abrupt start.

If plausible parameters representative of the breathing are not detected, only the base soundtrack is played.

Phase Shift/Desynchronizationloss of Signal

Continuously during playing of the back-and-forth soundtrack, the parameters representative of the breathing continue to be evaluated. It is evaluated whether the back-and-forth soundtrack remain sufficiently synchronized with the breathing. If, during a time greater than a preset time (e.g. 10 seconds), the back-and-forth soundtrack which is played is not sufficiently synchronized with the breathing, such as it is determined (for example because a phase of the breathing is difficult to determine during a preset time), the ratio of the amplitudes between the background soundtrack and the back-and-forth soundtrack is increased. Then, a new back-and-forth soundtrack is played, superposed on the base soundtrack and synchronized with the breathing. The prior back-and-forth soundtrack is progressively attenuated until it is no longer played.

As a variant, if there is access to the average breathing frequency with certainty but to the phase with uncertainty, breathing sounds could be played at the measured frequency without considering the phase shift.

As a variant, during the later use of the system (for example during a later night), the transition to later phases, compared to the previous night, is anticipated (whether by the time adjustment or depending on the falling-asleep metric). In fact, with use, the user is conditioned such that the transition to later phases guides them to falling asleep. Thus a neuronal feedback to the user (or, according to the term commonly used in the art, “neurofeedback”) is provided.

As a variant, a detection of the position of the user could be considered for re-adapting the measured phase offset. In this example, the position of the user is determined from data provided by the accelerometers. A database storing an estimated value of the phase shift between the actual breathing and the breathing measurement by the accelerometer as a function of the position (notably whether the user is lying on the back, stomach or side) is available. This preset phase shift is considered for the playing of acoustic signals.

As a variant, cognitive sounds related to the theme can be played randomly.

As a variant, the introductory phase is not played again if the functionality is restarted.

### Example 5: Guided Breathing

Starting the Functionality:

The user starts their night, with the “Guided Breathing” functionality for the falling asleep parameter and chooses a theme (e.g. nature). According to this functionality, a sound corresponding to inhaling is preselected. A sound corresponding to exhaling is preselected.

A background soundtrack related to the theme starts after a few seconds and lasts for a predetermined time, for example between 20 seconds and one minute (during which the breathing rhythm is detected-see the previous example for the measurement of parameters representative of the breathing).

Next, various sound signals are played superposed on the base soundtrack. To start, voice instructions are sent. For example, the following phrase is played: “—Breathe normally. When you hear the following sound, breath in,” then the sound corresponding to inhaling is played. Then, the voice instructions resume. The following phrase is played: “When you hear the following sound, breathe out.” Then, the sound corresponding to exhaling is played. Then, the voice instructions resume: “—Your breathing will progressively slow down until you are ready to sleep. At the right time, let go and allow your breathing to find its natural rhythm.”

First Chase of Breathing

Once the parameters representative of breathing are detected, and the voice instructions given, a back-and-forth soundtrack with a period corresponding to the period measured in the user is played (see example 4 on this subject). The back-and-forth soundtrack can in particular be superposed on the base soundtrack throughout the functionality. The sounds corresponding to inhaling and exhaling are played, superposed on the back-and-forth soundtrack, in synchronization with the breathing.

If the breathing frequency is not sufficiently reliably detected (see example 4), the first back-and-forth soundtrack is selected in a preset way, for example with an inhaling length and exhaling length equal to 3 seconds.

Going Down by Levels

In parallel with playing sound, the measurement of parameters representative of the breathing continues. The representative parameters measured are compared to the frequency and phase of the back-and-forth soundtrack just played. When the user follows correctly (meaning with a gap between the measured parameters and the parameters of the back-and-forth soundtrack less than a predetermined threshold) the back-and-forth soundtrack is played for several successive breaths (a preset number or a preset time), the period of the following back-and-forth soundtracks is increased (for example in two steps, T[n+1]=T[n]+025 s, T[n+2]=T[n+1]+0.25 s). The frequency of playing the sounds corresponding to inhaling and exhaling is also modified in the same way.

If the breathing is not detected, at the end of some preset time (for example 1 minute) the playing still goes down to the lower level.

Going Up by Levels

The synchronization of the breathing rhythm with the soundtrack is continuously detected. When it is detected that the user is behind compared to the sound indicators played, the period is reduced (the soundtrack is therefore accelerated) to allow the user to re-synchronize.

Stopping Functionality:

Four options can be provided for ending the functionality: the falling asleep metric indicator exceeds a preset threshold, the functionality was used for a preset time, the breathing is stabilized (see below), the breathing picks up (see below).

Stabilized Breathing:

If the user's breathing is stabilized at a lower level for a preset time, the back-and-forth soundtrack stops, the indicator sounds are no longer played, and only the background track continues to play, until interruption of the functionality.

The Breathing Picks Up:

If the breathing accelerates and is faster than the setting for a time greater than a preset time, the back-and-forth soundtrack stops, the indicator sounds are no longer played, and only the background track continues to play, until interruption of the functionality.

Forced Descent.

As a variant, if it is observed that the breathing is stable and very different from the breathing frequency of the level, the level can be lowered in sequence until reaching the measured frequency without necessarily following the rules for moving from one level to another described above.

As a variant, during the later use of the system (for example during a later night), the transition to later phases, compared to the previous night, is anticipated (whether by the time adjustment or depending on the falling-asleep metric). In fact, with use, the user is conditioned such that the transition to later phases guides them to falling asleep. Thus a neuronal feedback to the user (or, according to the term commonly used in the art, “neurofeedback”) is provided.

Rephasing.

As a variant, if the breathing is stable but in phase opposition for a long time, the breathing can be rephased by adding a silence of order the phase offset between two representative signals of consecutive inhaling and exhaling.

Breathing Training Sound.

Since the functionality ask for an action from the user, it is conceivable to make it the most natural possible. Consequently, as a variant, the sound will be chosen to be the most guiding possible for the user.

As a variant, cognitive sounds related to the theme played randomly will be used.

As a variant, the introductory phase is not played again if the functionality is restarted.

Interrupting the Functionality:

Various examples of functionalities are described above. Below, various examples of ending the functionality are described.

Interrupting the Functionality by Falling Asleep:

If the user reaches a certain stage of falling asleep, in a first step, the end determination module 23 orders the volume of the sounds (as applicable) to decrease over a preset time, for example of order 1 to 2 minutes. When the user is sufficiently asleep, the volume of the soundtrack next decreases over a preset time, for example of order 1 to 2 minutes.

Interrupting the Functionality by Timing Out:

At the end of a preset time, the functionality stops.

Restarting the Functionality:

After falling asleep, a pause ordered by the user or timing out, the functionality can only restart from the beginning.

Changing Theme

In case of changing theme via the portable computer user interface, the functionality restarts from the beginning.

Volume

The volume can be changed during the functionality via the portable computer user interface. The volume changes both the soundtracks and the sounds.

References:

system 1
support elements 2
module for acquisition 3 of at
least one measurement signal
reference electrode 3a
EEG measurement electrode 3b
bulk ground electrode 3c
playing module 4
two acoustic transducers 4a, 4b
processing module 5
module for providing an acoustic
stimulation sequence signal 6
storage module 6a
communication module 6b
battery 8
portable computer 9
wired connections 10
composition module 11
acoustic stimulation sequence 12
soundtrack database 13
sound database 14
pairing module 15
template database 16
template 17
falling-asleep metric
determination module 18
bank of reference data 19
bank of data 20
bank of reference data 21
representative of spectral
content indicator signals over
time representative of falling
asleep
selection module 22
end determination module 23
phrases database 24
server 100
measurement signal S

1. A wearable electronics system for helping a user fall asleep comprising:
a wearable portion suited to clothe the user;
an electronic portion comprising, functionally connected to each other;
at least one acquisition module suited for making a physiological measurement of the user;
at least one playing module suited for playing an acoustic stimulation to the user;
a memory storing a plurality of elemental acoustic stimulation sequences;
a processing module comprising a selection module suited for, repetitively during a period of falling asleep, selecting at least one elemental acoustic stimulation sequence from the plurality of elemental acoustic stimulation sequences stored in memory, depending on a history of previously played acoustic stimulations and depending on the physiological measurement, where the playing module is ordered to play said acoustic stimulation sequence, until the end of the period of falling asleep;
where at least the acquisition module and the playing module are assembled in the wearable portion.

2. The wearable electronic system according to claim 1, wherein the end of the period of falling asleep is determined by an end determination module of the processing module based on the physiological measurement.

3. The wearable electronic system according to claim 1, wherein the electronic portion generates, by repeated use of the selection module, an acoustic stimulation comprising a temporal succession of acoustic stimulation sequence signals, and in which the temporal succession is determined by the selection module depending on the history of previously played acoustic stimulations and/or depending on the physiological measurement.

4. The wearable electronic system according to claim 3, wherein a composition module generates an acoustic stimulation sequence signal based on a preset acoustic stimulation sequence template coming from a template database.

5. The wearable electronic system according to claim 3, wherein the composition module generates an acoustic stimulation sequence signal by superposition of several elemental acoustic stimulation sequences.

6. The wearable electronic system according to claim 5, wherein the elemental acoustic stimulation sequences are generated from databases of long length sound tracks, short length sounds, words or scenes, and phrases.

7. The wearable electronic system according to claim 6, comprising a module for pairing sound tracks, sounds, words or scenes.

8. The wearable electronic system according to claim 1, further comprising a portable computer proposing an interface interacting with the processing module.

9. The wearable electronic system according to claim 1, wherein the acquisition module is suited for making a physiological measurement of an acceleration, a movement, a heartbeat, a breathing cycle, a blood oxygen saturation or an electroencephalogram of the user.

10. The wearable electronic system according to claim 1 wherein the selection module selects an elemental acoustic stimulation sequence based on a falling-asleep metric estimator determined by a falling-asleep metric determination module based on the physiological measurement.

11. The wearable electronic system according to claim 1, wherein a target state indicator is determined, and the selection module selects an elemental acoustic stimulation sequence from a database relating a possibility of reaching the targeted state indicator for the user and the elemental acoustic stimulation sequences.
